(19) United States
(12) Patent Application Publication
(10) Pub. No.: US 2004/0110305 A1
(43) Pub. Date: Jun. 10, 2004

(54) Method of mixing a sample with a reagent in a chemistry

(76) Inventor: James Samsoondar, Cambridge (CA)

Correspondence Address: HOVEY WILLIAMS LLP, 2405 GRAND BLVD., SUITE 400, KANSAS CITY, MO, 64108, US

(21) Appl. No.: 10/724,249

(22) Filed: Nov. 26, 2003

Related U.S. Application Data
(62) Division of application No. 10/141,377, filed on May 7, 2002.
Application No. 10/141,377 is a division of application No. 09/664,704, filed on Sep. 19, 2000, now abandoned.

(51) Int. Cl. G01N 33/00

(52) U.S. Cl. 436/86

(57) ABSTRACT

An apparatus and method for performing immunoturbidimetric measurements of plasma proteins on an apparatus used for measuring plasma and serum interferents are described. Immunoturbidometric measurements are made on a sample in a disposable dispensing tip which acts as a cuvette and reaction chamber. These features allow test which are not available on general chemistry analyzers, to become available, and at the same time the apparatus can provide a screening system for serum and plasma interferents.

### FIELD OF INVENTION

This invention relates to immunoturbidimetry and spectrophotometric analysis of plasma for proteins.

### BACKGROUND OF INVENTION

Clinical laboratory tests are routinely performed on serum or plasma of whole blood. In a routine assay, red blood cells are separated from plasma by centrifugation, or red blood cells and various plasma proteins are separated from serum by dotting prior to centrifugation.

Haemoglobin (Hb), bilirubin (BR), biliverdin (BV), and light-scattering substances like lipid particles are typical substances which will interfere with and affect spectrophotometric and other blood analytical measurements. Such substances are referred to generally, and in this specification as interferents. Elevated BR and BV referred to as bilirubinemia and biliverdinemia respectively can be due to disease states, increased lipid particles in the blood also known as lipemia, can be due to disease states and dietary conditions; elevated Hb in the blood known as haemoglobinemia can be due to disease states and as a result of sample handling.

Many tests conducted on plasma or serum samples employ a series of reactions which terminate after the generation of chromophores which facilitate detection by spectrophotometric measurements at one or two wavelengths. Measurement of the quantity of interferents in a sample prior to conducting such tests is important in providing meaningful and accurate test results. In fact if a sample is sufficiently contaminated with interferents, tests are normally not conducted as the results will not be reliable.

Current methods used for detecting haemoglobinemia, bilrubinemia and lipemia or turbidity utilize visual inspection of the sample with or without comparison to a color chart. Visual inspection is sometimes employed on a retrospective basis where there is a disagreement between test results and clinical status of the patient in order to help explain such discrepancies.

Pre-test screening of samples by visual inspection is semi-quantitative at best, and highly subjective and may not provide sufficient quality assurance as required for some tests. Furthermore, visual inspection of samples is a time consuming, rate limiting process. Consequently, state-of-the-art blood analyzers in fully and semi automated laboratories do not employ visual inspection of samples.

Other methods used to assess the amount of contamination of a sample, i.e., sample integrity, employ direct spectrophotometric measurement of a diluted sample in a special cuvette. In order to obtain a measurement of the sample of the plasma or serum, sample tubes must be uncapped, a portion of the sample taken and diluted prior to measurement. Both of these steps are time consuming and require disposable cuvettes.

An apparatus used for measuring sample integrity can also be used to measure plasma proteins, e.g., Immunoglobulin A (IgA), β2-microglobulin and C-reactive protein (CRP). To do so, an antibody reagent is required for each protein, and a 37° C. incubation chamber. This method of analysis is called immunoturbidimetry because the specific antibody reagent forms immunocomplexes with the corresponding protein, when present in the sample. The immunocomplexes scatter light in various directions depending on the size distribution of the immunocomplexes or particles; turbidity in a sample is a result of scattered light and the absorbance increase is inversely proportional to wavelength. It must be understood that the use of the term absorbance includes “true absorbance” and the effect of light loss by any other means; the detector in the spectrometer measures the light transmitted through the sample, and absorbance is calculated as the negative log of transmittance. Therefore, any light which does not reach the detector, e.g., due to scattering caused by turbidity, will be interpreted as absorbed light.

For proteins in low concentrations, e.g., in the order of mg/L, the turbidity created by immunocomplexes is very small and are usually measured in one of two ways: 1) Measurement of light scattered in the forward direction on an instrument called a nephelometer, which is like a spectrophotometer that measures light propagated at an acute angle to the incident light. Such a method would require a separate instrument which would increase the cost per test; 2) Measurement of “absorbance” at 340 nm by a spectrophotometer. In the prior art which uses absorbance measurements, the absorbance at 340 nm at zero time is subtracted from the absorbance at 340 nm after incubation at approximately 37° C. for approximately five minutes, in order to remove the effect of sample interferents. This approach cannot be used for the near infrared (NIR) and adjacent visible wavelengths where the light-scattering caused by the immunocomplexes is very small.

### SUMMARY OF THE INVENTION

It is desirable to use an apparatus designed for measuring plasma and serum interferents to perform immunoturbidimetric measurements. This feature allows tests which are not available on general chemistry analyzers, to become available, and at the same time the apparatus can provide a screening system for serum and plasma interferents.

The present invention uses a novel wavelength range and method to subtract endogenous sample turbidity and the effect of other interferents. The present invention uses a disposable dispensing tip in a novel way both as a reaction and incubation chamber, as well as a cuvette. The use of a disposable dispensing tip as a reaction chamber and cuvette allows this invention to be integrated into a chemistry analyzer, or built as a stand-alone instrument f r measuring serum and plasma interferents as well as plasma proteins. This invention is particularly relevant to chemistry analyzers which do not already possess similar optical hardware as described for this invention, which could facilitate the measurement of serum and plasma interferents, and plasma proteins. By integrating such optical capabilities in the chemistry analyzer, the current test menu can be expanded by offering immunoturbidimetric measurements.

Accordingly, the present invention provides an apparatus for determining the concentration of one or more plasma proteins in a sample by immunoturbidimetry, said apparatus comprising:

a blood analyzer;

a disposable dispensing tip;

means for sealing a first end of the disposable dispensing tip;

a second tip capable of being inserted into an open second end of the disposable dispensing tip for adding one or more reagents to the disposable dispensing tip;

a heated cavity for receiving the sample in the disposable dispensing tip of the analyzer;

means for transferring the disposable dispensing tip into and out of the heated cavity;

a radiation source for emitting a beam of radiation;

means for directing the radiation onto the sample in the disposable dispensing tip;

a sensor responsive to receipt of the radiation; and

means for correlating said concentration of the one or more proteins in the sample to a sensor response from the sample. Preferably the means for sealing is a vice and the radiation source means, means for directing said radiation onto said sample, and sensor are contained in a spectrophotometer. More preferably the beam of radiation is near infrared and adjacent visible region light and has wavelengths from about 475 nm to about 910 nm.

An apparatus of the invention for the correlation referred to above incorporates calibration algorithms in respect of IgA, β2-microglobulin and C-reactive protein (CRP) respectively which are:

mg/L IgA=−a(X nm)+b(Y nm)−c a.

where a, b and c are coefficients of the first derivative of absorbances at the wavelengths X and Y; (X nm) is the first derivative of the absorbance at wavelength X; (Y nm) is the first derivative of the absorbance at wavelength Y; preferably a=3327100-3327120, b=484250-484290 and c=70-85, more preferably a=3327114.33, b=484270.80 and c=77.3; where X is about 780-800 nm, and Y is about 820-830 nm, preferably X is about 789 nm and Y is about 825 nm

mg/L β2-microglobulin=a(X nm)+b(Y nm)+c b.

where a, b and c are coefficients of the first derivative of absorbances at wavelengths X and Y; (X nm) is the first derivative of the absorbance at wavelength X; (Y nm) is the first derivative of the absorbance at wavelength Y; preferably a=−33640-33660, b=36550-36560 and c=2-3, more preferably a=−33648.79, b=36556.81 and c=2.3; where X is about 545-550 nm and Y is about 825-835 nm, preferably X is about 548 nm and Y is about 829 nm;

mg/L CRP=a(X nm)+b(Y nm)+c c.

where a, b and c are coefficients of the first derivative of absorbances at wavelengths X and Y; (X nm) is the first derivative of the absorbance at wavelength X; (Y nm) is the first derivative of the absorbance at wavelength Y; preferably a=(−1813675)-(−1813685), b=1808670-1808680 and c=9.5-10, more preferably a=−1813682.71, b=1808677.58 and c=9.8; where X is about 655-665 run and Y is about 675-685 run, preferably X is about 661 nm and Y is about 679 nm.

In another aspect the invention, there is provided a method for determining the concentration of one or more plasma proteins in a sample by immunoturbidimetry in a blood analyzer, the method comprising:

filling a disposable dispensing tip with the sample;

sealing a first end of the tip with means for sealing;

adding a reagent to an open second end of the disposable dispensing tip with a second tip capable of being inserted into the open end;

placing the disposable dispensing tip into a heated cavity;

radiating the sample in the disposable dispensing tip with a source which emits a beam of radiation;

sensing the radiation having passed through the sample;

correlating the concentration of said one or more proteins in said sample to the sensor response from the sample. The disposable dispensing tip which contains the reagent or reagents and sample may be removed from the heated cavity prior to being subjected to radiation. The preferred means for sealing is a vice. The method also contemplates that the beam of radiation is near infrared and adjacent visible region light, preferably the near infrared and adjacent visible region light has wavelengths from about 475 nm to about 910 nm.

Concerning this method the correlation referred to above incorporates calibration algorithms in respect of IgA, β2-microglobulin and C-reactive protein (CRP) respectively which are:

mg/L IgA=−a(X n)+b(Y nm)−c a.

where a, b and c are coefficients of the first derivative of absorbances at the wavelengths X and Y; (X nm) is the first derivative of the absorbance at wavelength X; (Y nm) is the first derivative of the absorbance at wavelength Y; preferably a=3327100-3327120, b=484250-484290 and c=70-85, more preferably a=3327114.33, b=484270.80 and c=77.3; where X is about 780-800 nm, and Y is about 820-830 nm, preferably X is about 789 nm and Y is about 825 nm

mg/L β2-microglobulin=a(X nm)+b(Y nm)+c b.

where a, b and c are coefficients of the first derivative of absorbances at wavelengths X and Y; (X nm) is the first derivative of the absorbance at wavelength X; (Y nm) is the first derivative of the absorbance at wavelength Y; preferably a=−33640-33660, b=36550-36560 and c=2-3, more preferably a=−33648.79, b=36556.81 and c=2.3; where X is about 545-550 nm and Y is about 825-835 nm, preferably X is about 548 nm and Y is about 829 run;

mg/L CRP=a(X nm)+b(Y nm)+c c.

where a, b and c are coefficients of the first derivative of absorbances at wavelengths X and Y; (X nm) is the first derivative of the absorbance at wavelength X; (Y nm) is the first derivative of the absorbance at wavelength Y; preferably a=(−1813675)-(−1813685), b=1808670-1808680 and c=9.5-10, more preferably a=−1813682.71, b=1808677.58 and c=9.8; where X is about 655-665 nm and Y is about 675-685 nm, preferably X is about 661 nm and Y is about 679 nm.

The present invention also provides a method for determining the concentration of plasma protein IgA, β2-microglobulin or C-reactive protein in a plasma sample by immunoturbidimetry in a blood analyzer, said method comprising:

aspirating a small volume of plasma into a disposable dispensing tip;

further aspirating the small sample in the sample tip to pull the sample away from the lower end of the tip;

sealing the lower end of the tip with means for sealing the tip without trapping air below the sample in the tip;

adding an antibody reagent to the disposable dispensing tip with a second dispensing tip, the second tip is capable of being inserted into the open end of the disposable dispensing tip;

heating the disposable dispensing tip in a heating cavity;

radiating the sample in a disposable dispensing tip in a spectrophotometer; and

correlating the concentration of the IgA, β2-microglobulin or C-reactive protein in the sample to a sensor response from the sample. Preferably the temperature of the heating cavity is 37° C. and the tip is maintained in a heating cavity for 2 minutes. More preferably, the plasma sample is 5 μl. In a preferred embodiment the antibody reagent is about 60 μl of antibody selected from the group consisting of: antibody reactive to IgA; antibody reactive to β2 microglobulin; and antibody reactive to C reactive protein.

### DESCRIPTION OF THE DRAWINGS

FIG. 1 is a perspective view of a system incorporating an apparatus of the present invention for analyzing sample integrity and measuring a variety of proteins;

FIG. 2 is a schematic representation of elements of the apparatus of FIG. 1;

FIG. 3 is a perspective view of two disposable dispensing tips and jaws of a small vice used to squeeze the lower end of the tip, for the purpose of sealing;

FIG. 4 is a graphic representation of the absorbance spectra of variable amounts of IgA, zero time after incubation with antibodies against IgA, at 37° C., in the dispensing tip of an analyzer. The concentration of IgA is shown in the figure;

FIG. 5 is a graphic representation of the absorbance spectra of variable amounts of IgA, 2 minutes after incubation with antibodies against IgA, at 37° C., in the dispensing tips of an analyzer. The concentration of IgA is shown in the figure;

FIG. 6 is a graphic representation of a linear regression fit gor data used for the development of an IgA calibration algorithm for samples in dispensing tips of an analyzer, with IgA in units of milligrams per litre on the abscissa and ordinant axes;

FIG. 7 is a graphic representation of a linear regression fit for data in respect of predicted IgA concentration for samples not used in the calibration process, for samples in dispensing tips of an analyzer, with IgA in units of milligrams per litre on the abscissa and ordinant axes;

FIG. 8 is a graphic representation of a linear regression fit for data used for the development of a β2-microglobulin calibration algorithm for samples in dispensing tips of an analyzer, with β2-microglobulin in units of milligrams per litre on the abscissa and ordinant axes;

FIG. 9 is a graphic representation of a linear regression fit for data used for the development of a C-reactive protein calibration algorithm for samples in dispensing tips of an analyzer, with C-reactive protein in units of milligrams per litre on the abscissa and ordinant axes;

FIG. 10 is a graphical representation of the percent error in IgA prediction caused by endogenous turbidity created by intralipid, with and without subtraction of the 1st derivative of the absorbance at zero time.

### DESCRIPTION OF THE INVENTION

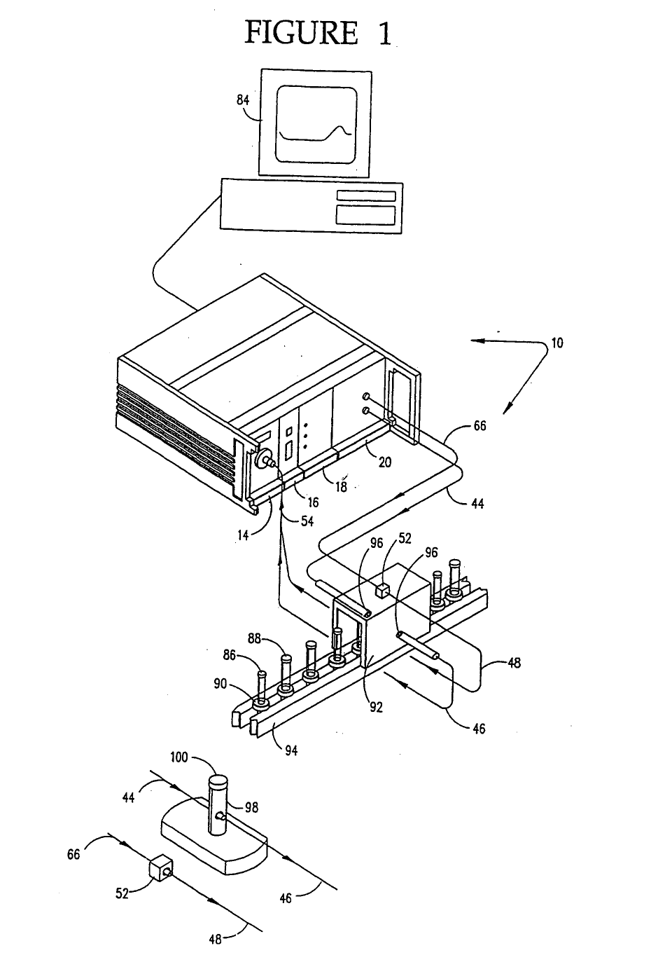

As discussed above, the present invention provides apparatus and a method for performing immunoturbidimetric measurements on an apparatus used for measuring plasma and serum interferents. This feature allows tests which are not available on general chemistry analyzers, to become available, and at the same time the apparatus can provide a screening system for serum and plasma interferents. The apparatus for measuring serum and plasma interferents comprises a housing for receiving a sample; a radiation source; a sensor; a means for optically connecting the radiation source with the sensor along a sample path through the housing and along a reference path which bypasses the sample; a means for selectively passing a beam from the sample path and from the reference path to the sensor; and a means for correlating a sensor response, from the sample path relative to a sensor response from the reference path, to a quantity of a known substance in said sample. The sample housing can be an integral part of the conveyor system as shown in FIG. 1, or the housing can have a cavity for receiving a sample and a lid for selectively opening and closing the cavity, also shown in FIG. 1. A cover may not be necessary in an automated system, where the dispenser stem, when inserted into the dispensing tip, can provide sufficient light shielding, and further because of the strategic location of the shutters, the subtraction of dark current from both the sample and the reference light measurements, can effectively eliminate the effect of room light. The radiation source is for emitting a beam of radiation, and the sensor is responsive to receipt of radiation. In order to perform immunoturbidimetry using an existing apparatus, a means for sealing the lower end of the dispensing tip as required. In a preferred embodiment the means for sealing is a small vice. A preferred example of a dispensing tip is the disposable tip used by the Vitros™ analyzer manufactured by Johnson and Johnson.

The apparatus further comprises a quartz-tungsten-halogen lamp capable of emitting a near infrared, and adjacent visible region light beam having wavelengths from 475 nm to 910 nm and a bifurcated fibre-optic cable for splitting the light beam from the quartz-tungsten-halogen lamp into a sample path beam for travel along a sample path and a reference path beam for travel along a reference path. This apparatus further comprises a shutter for selectively blocking the sample path light beam which travels along the sample path and the reference path light beam which travels along the reference path, as well as optical fibre bundles for transmitting the sample path light beam through a sample enclosed in the housing, and optical fibre bundles for transmitting the sample path light beam from the sample to a second bifurcated fibre-optic cable, where the beam from the sample path and the beam from the reference path converge into a single fibre-optic cable. It is understood that any means for excluding from the sample, light other than that from the radiation source of the apparatus, is within the scope of this invention. Also, if dark current, i.e., sensor response when sensor is not exposed to the instrument light, is subtracted from both the reference and sample measurements, the room light impinging on the detector can be effectively subtracted without affecting the instrument performance significantly.

Preferably, the bottom end of the dispensing tip is sealed by flattening between the jaws of a small vice, after a sample is aspirated into said tip. Preferably the dispensing tip is disposable and more preferably the tip of an analyzer is used as a reaction and incubation chamber after the tip is sealed with the sample inside, and the same sealed dispensing tip is used as a cuvette.

Analytes, such as proteins, preferably Immunoglobulin A (IgA), β2-microglobulin and C-reactive protein (CRP), can be measured on the apparatus through the use of reagents, eg. antibodies, by the process of immunoturbidimetry. Each plasma protein requires a specific antibody, and the specificity of each test can be increased by subtracting the first derivative of the absorbance at zero time from the first derivative of absorbance after approximately 2 minutes at 37° C., at single or multiple wavelengths. It will be understood that optimum incubation time and temperature may vary for different plasma proteins.

Only 5 μL of sample and 60 μL of antibody reagent is required. It will be understood that optimum sample and reagent volumes may vary for different proteins.

In another aspect of the invention, the same dispensing tip used to aspirate 5 μL of sample is sealed at the lower end by increasing the vacuum on the tip by an equivalent of 4 μL. It will be understood that deviations from this volume are within the scope of this invention, particularly when other disposable tips are used. The extra vacuum equivalent to an aspiration of 4 μL, is sufficient to pull the fluid away from the lower end of the tip which is within the grasp of the jaws of a small vice without trapping air below the 5 μL of sample, and without trapping sample below or within the seal.

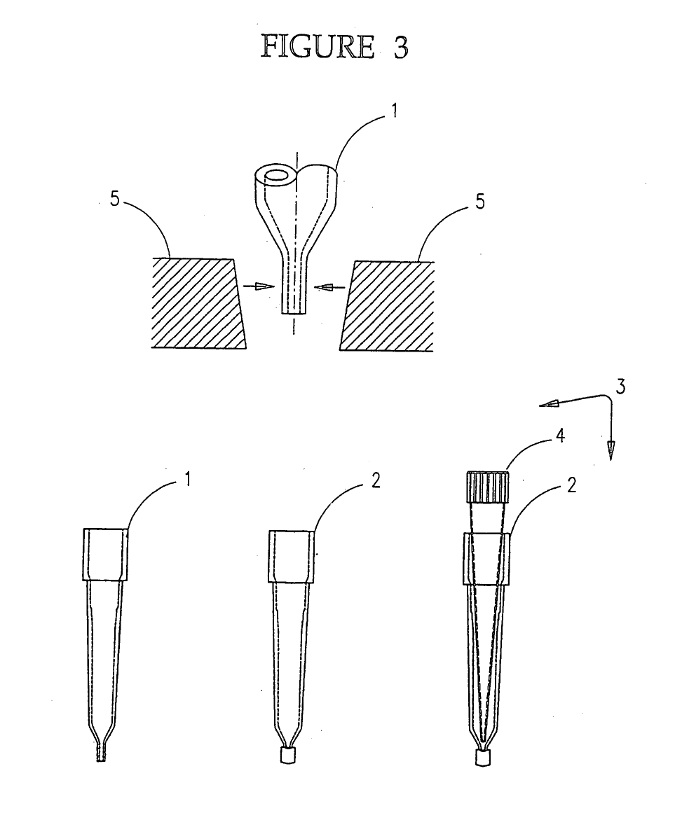

According to a preferred embodiment, the jaws are slightly nonparallel as shown in FIG. 3, and will therefore force upwards any residual fluid which is in the grasp of the jaws. This aspect of the invention assists in reducing any loss of any part of the sample.

In practising the invention, an antibody reagent is mixed with the sample by injecting 60 μL of antibody reagent into 5 μL of a sample. Preferably, 60 μL of antibody reagent is in a narrow pipette tip, e.g., as shown as 4 in FIG. 3, which can reach the bottom of the sealed tip, allowing enough space to facilitate proper dispensing of the antibody reagent. More preferably, narrow tips such as shown as 4 in FIG. 3 are 960 Envirotips® manufactured by Eppendorf, but any similar tip may be used. It is desirable that the ratio of antibody reagent volume to sample volume facilitates adequate mixing of sample and reagent. In carrying out the invention it is preferable if the ejection of the antibody reagent is such that only the fluid is ejected and no air is injected into the reaction chamber.

Zero-time absorbance measurement is triggered after antibody reagent is dispensed into a sealed tip of the invention, and the zero-time measurement is performed with the dispensing stem attached to the tip.

According to one embodiment of the invention, the tip holder has a sliding lid which closes after antibody reagent is dispensed.

In another aspect of the invention, because of the location of the shutters in the lamp assembly the subtraction of dark current from both the sample and the reference light measurements, can effectively eliminate the effect of room light. Preferably the sample chamber is shielded from light but is not required to be completely light-tight; a cover may n t be necessary in an automated system, where the dispenser stem, when inserted into the dispensing tip, can provide sufficient light shielding, even when dark current is not subtracted.

In another embodiment of the invention, the spectrometer can be run in single-beam mode.

In another aspect of an alternative embodiment of the invention, zero-time measurement is used as the reference scan when the spectrometer is run in the single-beam mode. Preferably the rate of change of the first derivative of absorbance is monitored during the first 15 seconds, to forecast if a high-dose hook effect will occur.

Immunoturbidimetric measurements are performed using multiple wavelengths in the visible and NIR electromagnetic radiation.

A method of the invention provides for measuring the concentration of a series of proteins in a sample by recording the absorbance spectrum of the sample before and after incubation with antibodies specific to each protein. Preferably the effect of interferents in a sample can be minimized by virtue of the wavelength range used, i.e., NIR and adjacent visible radiation. More preferably the remaining effect of interferents can be substantially removed by subtracting the first derivative of absorbance at zero time from the first derivative of absorbance after a two-minute incubation at 37° C. It will be understood that other times and incubation temperatures can be used.

The effect of small air bubbles on absorbance is minimized by using the first derivative of absorbance. It will be understood that any higher order of derivative of absorbance may also be used, eg., second derivative of absorbance.

A system incorporating the apparatus of the present invention is generally illustrated in FIG. 1. The apparatus 10 generally comprises a spectrometer 14 optically coupled to, or communicating with a sample held on the conveyor 94 through fibre optic bundles 44 and 46, installed in a cover 92, or a sample holder 98 with a cover 100. Apparatus 10 is mounted or installed adjacent to an automated conveyor 94 which carries a plurality of sample tubes, e.g. 86 and 88. Because samples are presented in variable tube sizes, there may be a gap between the walls of the tube and the ends of fibres 44 and 46, focusing lenses 96 are attached to the ends of the fibres. Sample holder 98 is designed for a disposable dispensing tip. Cover 92 acts as a light shield and also provides a restraint for the fibres 44 and 46, against any movement.

Cover 100 in FIG. 1 also acts as a light shield for the apparatus. The dispensing stem of an analyzer and the tip holder can act as a light shield, with the tip holder designed deep enough to accommodate the stem of the analyzer dispenser. Neither the tip holder and cover 100, nor cover 92 are intended to provide a light-tight sample chamber. Sample presentation on a conveyor line 94 in FIG. 1 is nly relevant to the analysis of sample integrity functionality of the spectrometer. For the present invention, a sample is presented to the optical apparatus in a tip holder 98 in FIG. 1.

For the measurement of proteins by immunoturbidimetry, a separate sample holder such as that illustrated (98) is required, and is imbedded in a heated block. In a preferred embodiment, 5 μL of plasma is aspirated in a dispensing tip, as shown as 1 in FIG. 3. Extra vacuum, equivalent to an aspiration of 4 μL is applied to the sample to pull the fluid away from the lower end of the tip which is within the grasp of the jaws as shown in FIG. 3. Different volumes can be used, it being understood that the objective is to have the sample removed far enough from the tip and to allow for sealing. The extra vacuum must be sufficient to pull the fluid away from the lower end of the tip, without trapping air below the 5 μL of sample. The same dispensing tip used to aspirate 5 μL of sample is sealed after the sample is aspirated into said tip. The end of the dispensing tip is sealed underneath the 5 μL of sample by squeezing between the jaws of a small vice, shown as 5 in FIG. 3. The sealed tip with a flattened lower end is shown as 2 in FIG. 3. It will be understood that although FIG. 3 shows a Vitros™ tip as 1 and 2, other disposable tips can be used and deviations from 4 μL are within the scope of this invention, particularly when other disposable tips are used. The jaws 5 in FIG. 3 are slightly nonparallel, and will therefore force upwards, any residual fluid which is in the grasp of the jaws. This aspect of the invention precludes loss of any part or the 5 μL of the sample.

In this invention, analytes are measured on the apparatus through the use of reagents by the process of immunoturbidimetry. Each protein requires a specific antibody, and the specificity of each test can be increased by subtracting the first derivative of the absorbance at zero time from the first derivative of absorbance after approximately 2 minutes at 37° C., at a single or multiple wavelengths. It will be understood that optimum incubation time and temperature could vary for different proteins.

60 μL of antibody reagent is aspirated from a bottle into a narrow pipette tip shown as 4 in FIG. 3. In a preferred embodiment, narrow tips shown as 4 in FIG. 3 are 960 Envirotips® manufactured by Eppendorf, but any similar tip which can reach the bottom of the sealed tip may be used. The Eppendorf tip or its equivalent must be allowed to reach the bottom of the Vitros tip or its equivalent, with just enough space between the ejection port and the 5 μL of sample, to facilitate proper dispensing of the antibody reagent. The antibody reagent is mixed with the sample by injecting the 60 μL of antibody reagent into the 5 μL of sample. Little or no air should be injected into the sample. This can be accomplished by injecting the 60 μL or less of the antibody reagent, as long as the volume is dispensed in a precise manner. It will be understood that further mixing can be achieved by reaspirating and redispensing the reaction mixture.

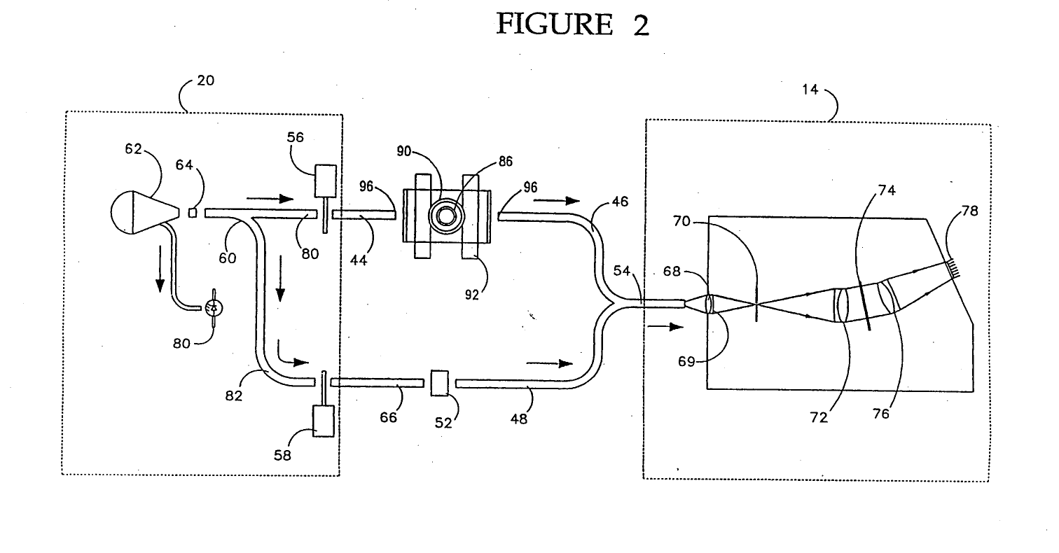

The disposable dispensing tip of an analyzer is used as a reaction and incubation chamber after the tip is sealed with the sample inside; it is also used as a cuvette. Although FIG. 1 only shows one tip holder 98, a preferred embodiment contains two tip holders 98; one used for measurement of interferents and the other for protein measurement. It will be understood that one tip holder can be used for both applications, and the single tip holder is heated for the benefit of the protein measurement, without affecting the interferent measurements, since the dwell time for the interferent measurement is only one second. When two separate tip holders are installed, they are connected through a bifurcated optical fibre, to the sample optical fibre 44 in FIG. 1. Two new shutters must be installed external to the lamp assembly 20 in FIGS. 1 and 2. The new shutters allow light to be directed only to the tip holder which is functional.

Zero-time absorbance measurement is triggered after the antibody reagent is dispensed, with the dispensing stem attached to the tip. In another embodiment of the invention, the tip holder has a sliding lid which closes after the antibody reagent is dispensed, and after the dispensing stem releases the tip. The effect of interferents can be substantially removed by subtracting the first derivative of the absorbance at zero time from the first derivative of absorbance after a two-minute incubation at 37° C. It will be understood that other times and incubation temperatures can be used. In this design, the sample holder functions as both the incubator and the optical read station. It will be understood that the incubation can occur in a separate chamber, where the incubated sample can be aspirated into a disposable dispensing tip, which is subsequently placed in the tip holder 98 as shown in FIG. 1. If a separate incubation chamber is used, the same read station or tip holder 98, as shown in FIG. 1, can be used for both interferent and protein measurements. If a combined incubator-read station is used, then a separate tip holder is required for measuring interferents, and a separate set of optical fibres and shutters are required to supply and receive radiation to and from the “incubator-read station”. If it is desired to have the dispensing stem remain with the dispensing tip, a second dispensing stem, can be added to the apparatus.

Sample fibres 44 and 46 direct radiation from a light source to and from the sample respectively, and allow the bulk of the instrumentation to be placed remotely from the samples. Multiple optical fibres 46 and 48 are the strands of a bifurcated optical fibre which collect radiation alternately from the sample 44 and reference optical fibre 66, and combines into one multiple optical fibre 54 which communicates with a spectrometer 14. Reference fibre 66 is joined to a strand 48 of the bifurcated fibre by a coupling 52. The coupling 52 can be chosen to provide sufficient attenuation of the reference beam, where the detector is optimally integrated over a short period of time. Fibre 66 is a single fibre and fibre 44 can be a single or multiple fibres, depending on the light throughput required.

Referring to FIG. 1, the apparatus 10 includes a spectrometer 14, a central processing unit 16, a power supply 18, a lamp assembly module 20 and a sample holder 92 and 94, or 98.

Referring to FIG. 2, the lamp assembly module 20 employs a light source 62. Preferably the light source is a 20-watt quartz-tungsten-halogen lamp, but other wattage lamps can be employed. The input power supply is alternating current, but the output to the light source is a stabilized direct current. Attached to the lamp is a photodiode 80, which monitors lamp output. Spectral output from light source 62 is a broad band covering visible and NIR regions. Although the NIR region of the electromagnetic spectrum is generally considered to be the interval extending from 650 nm to 2700 nm, the nominal wavelength range of the preferred embodiment is from 475 nm to 910 nm, which is referred to as the “near infrared and adjacent visible region”. A beam of radiation from the light source 62 is directed through a band-pass filter 64 and a shaping filter 69 in the spectrometer 14. The band-pass filter is required to reduce unwanted radiation outside of 475-910 nm. The shaping filter 69 is required to “flatten” the detection system's optical response. The beam of radiation from filter 64 is transmitted through a bifurcated optical multi-fibre bundle 60 to provide sample and reference beams. Bifurcated bundle 60 provides random sampling of lamp radiation to supply the sample and reference beams via two arms of 60, 80 and 82 respectively. In a preferred embodiment, a balanced emerging radiation is provided to the photo diode array (PDA) detector 78, from both the sample and reference paths, where the radiation through 80 and 82 are 99% and 1% respectively. With shutter 58 closed and shutter 56 open, radiation is channeled through optical fibre 44 to the sample, and the radiation transmitted through the sample in multiple-labeled tube or plastic dispensing tip and is received by fibre 46, which returns collected radiation to the spectrometer 14.

The sample and reference beams enter arms 46 and 48 respectively of a bifurcated optical multi-fibre bundle which combine in fibre 54 and are focused alternately onto a slit 70, by a focusing lens 68 and a shaping filter 69. Emerging radiation is collimated by lens 72 before the beam is directed to grating 74 which is a dispersing element which separates out component wavelengths in a preferred embodiment dichromated gelatin is used as the grating material. Component wavelengths are focused by a lens 76, onto the PDA 78. Each element or pixel of the PDA is set to receive and collect a predetermined wavelength. In a preferred embodiment the PDA comprises 256 pixels. The pixels are rectangular in shape to optimize the amount of optical radiation detected.

Spectrometer 14 is preferably a “dual-beam-in-time” spectrometer with fixed integration time for the reference beam and a choice of integration for the sample beam. Because the sample is only shielded from light, but is not in a light-tight holder, sample and reference dark scans can be subtracted from sample and reference light scans respectively; sample and reference dark scans are performed at the same integration times used for the respective light scans. In a preferred embodiment, the reference scan is performed at 13 milliseconds, and the sample scan is performed in 20 milliseconds; the maximum ADC value obtained at 20 milliseconds for a particular sample, is used to determine a new integration time up to 2600 milliseconds, such that saturation of the detector at any pixel does not occur. The maximum time allowed for any sample depends on the required speed of sample screening. Also, multiple scans can be averaged to minimize noise, but for interferent and protein measurements, the number of scans averaged must not require more than 1 second.

When in use, each pixel or wavelength portion is measured approximately simultaneously during a particular scan. Optical radiation falling on each sensor element is integrated for a specified time and individual pixels or wavelengths are samples sequentially by a 16 bit analog-to-digital convertor or ADC.

Although the present embodiment details use of a PDA, any alternative means which achieves the same result is within the scope of the present invention. For example a filter-wheel system may be used. In carrying out measurements each analyte uses from one to three wavelengths or pixels. Given that the first derivative of absorbance with respect to measurements with the PDA is the difference between the absorbance at two adjacent pixels, the first derivative of absorbance at one wavelength with a filter-wheel system will require absorbances measured with two different narrow band-pass filters. It will be readily understood by those skilled in the art that the filters do not need to be assembled on a rotating wheel, but that any structure which achieves the result of a narrow band-pass filtration of absorbed radiation is within the scope of the present invention.

The PDA integrates the optical radiation over a specified time and converts the optical signal to a time multiplexed analog electronic signal called scan where absorbance is calculated as:

Absorbance=log(Referenceᵢ/Sample measurementᵢ)+log(ITM/ITR)

where Referenceᵢ=reference pixel i readings;

Sample measurementᵢ=sample measurement pixel i reading;

ITM=Integration time measurements;

ITR—integration time reference; and

i=the particular pixel in the PDA.

In respect of these calculations, absorbance can also equal log (reference—reference dark measurement}/{sample measurement−sample dark measurement})+log(ITM/ITR)

Depending upon the amount of light shielding provided by the apparatus and the criticality of timing, the measurement of a reference dark and sample dark values may or may not be undertaken. The electronic signal is proportional to the time that the sensor integrates the optical signal. The electronic signal is amplified by analog electronic amplifiers and converted to a digital signal by an analog-to-digital converter or ADC. The digital information from the converter is interpreted for data analysis by a microprocessor which is in turn connected via an RS232 connector to a computer 84. The results of the data analysis can be shown on an output device such as a display and on a printer.

The first part of the process for generating a calibration curve is to store spectral data for the calibration set. The calibration algorithm for each protein must be installed in a microprocessor so that when an unknown sample is tested for a particular protein the result is quickly produced in order to calculate the quantity of any protein present, any one of several different methods, all of which are within the scope of this invention, may be used.

A preferred method is to calculate the first derivative of certain portions of the spectra in respect of the particular protein being measured. It is also possible to calculate the second, or third derivatives, and such calculations are within the scope of this invention. However, each step of taking differences to calculate those derivatives is more time consuming and introduces more noise.

In practice, an optimal combination of first derivatives of at least two portions of a spectrum generated from a scan for a particular protein are used to calculate protein concentration. The precise approach used depends on the protein being measured.

### EXAMLES

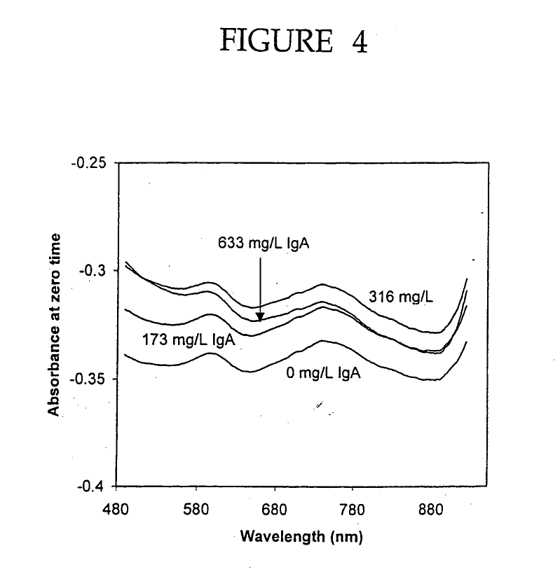

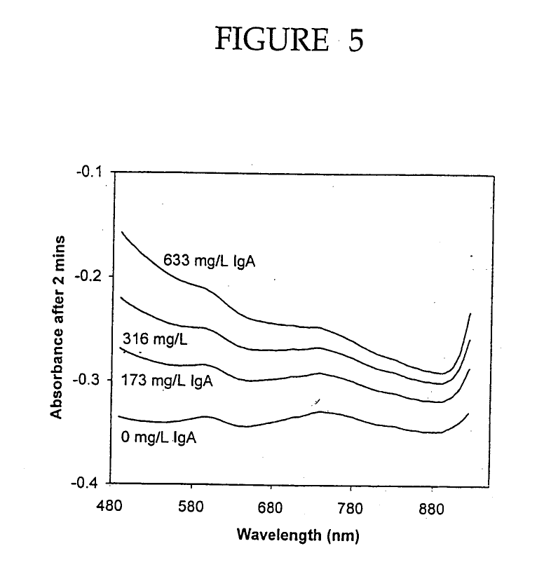

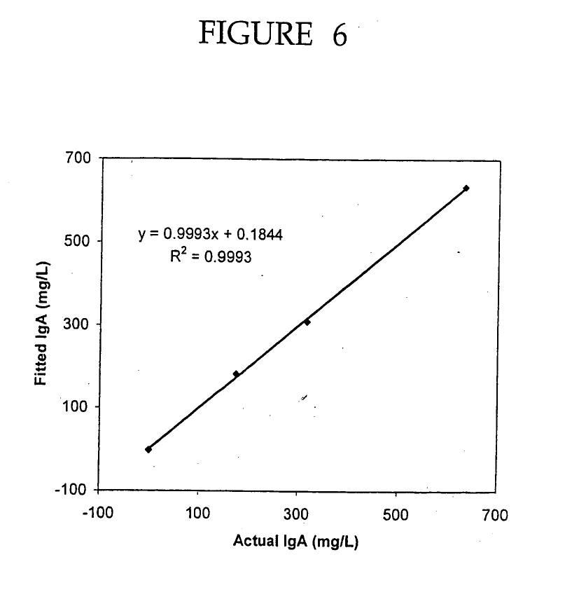

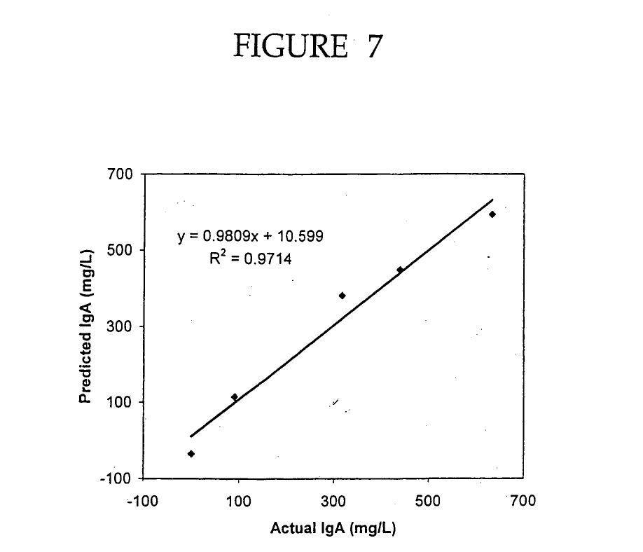

With respect to generating a calibration curve for IgA, 5 μL of each calibrator was aspirated in a Vitros dispensing tip using an Eppendorf pipette. The pipette setting was changed from 5 μL to 9 μL; this extra vacuum allowed the sample to be drawn away from the end of the tip which is within the grasp of the vice shown in FIG. 3. In order to prevent the fluid from leaking out, the bottom end of the dispensing tip was sealed by squeezing it with a pair of pliers. The tip with the fluid was placed in the heated tip holder, shown as 98 in FIG. 1. Using a second pipette, 60 μL of antibody reagent was added to the sample, with the lower end of the Eppendorf pipette tip almost in contact with the sample, as shown as 3 in FIG. 3. The Eppendorf tip must reach as far down as possible, without restricting the flow of the antibody reagent. Immediately after the antibody reagent is added, the absorbance spectrum was recorded as the zero-time measurement. Two minutes later, a second absorbance spectrum was recorded. This was repeated for the 4 calibrators, and 5 independent samples used for validation of the developed calibration algorithm. The absorbance spectra for the calibrators and validation sample set are shown in FIGS. 4 and 5 respectively. The linear regression fit for the calibrators and validation sample set are shown in FIGS. 6 and 7 respectively.

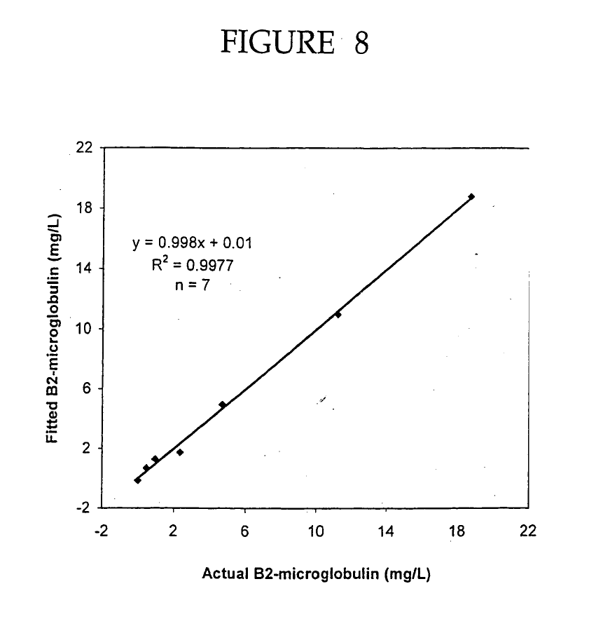

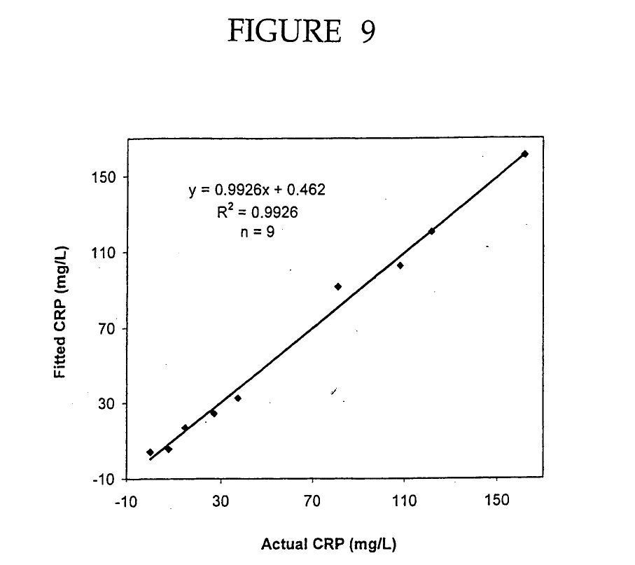

Similarly, calibration algorithms were developed β2-microglobulin and C-reactive protein, and their linear regression fits are shown in FIGS. 8 and 9 respectively. The antibody used for β2-microglobulin is covalently coupled to polystyrene beads in order to and the antibody used for CRP was unenhanced, like the IgA antibodies. These antibodies are also available commercially. It must be understood that any protein for which specific antibodies are available, and where the concentration is sufficient to develop detectable immunocomplexes, can be measured by this invention. Furthermore, for proteins in relative low concentrations, the signals can be enhanced by coupling polystyrene beads to the antibody.

Due to the small absorbances which is expected at the wavelengths used, the zero-time absorbance spectra obtained for IgA were observed to be in a random order, as shown in FIG. 4, possible due to the presence of tiny air bubbles in the fluid and inconsistencies in the walls of the dispensing tip. However, after 2 minutes at 37° C., both the absorbances and the first derivative of the absorbance are proportional to the concentration of β2-microglobulin, as shown in FIG. 5. The prior art subtracts the zero absorbance at around 340 nm, from the absorbance at 340 nm after the incubation at approximately 37° C. for approximately 5 minutes, for the purpose of removing the effects of interferents in the sample. To those skilled in the art, the use of dispensing tips along with the prior art method to remove the effects of interferents cannot be use for the wavelength range as specified in this invention. The present invention uses a new approach for removing the effects of interferents, where the first derivative of absorbance is subtracted from the first derivative of absorbance after 2 minutes at 37° C. at every wavelength; the difference is then subjected to a statistical process of step-wise linear regression for the selection of optimal wavelengths. It will be understood that for the calculation of each first derivative of absorbance in the preferred embodiment, requires the raw absorbances at 9 pixels or wavelengths; if filters were used instead of the PDA used in this invention, 2 narrow band-pass filters would be required to produce each first derivative of absorbance. Therefore, even if a single first derivative of absorbance is used in the calibration algorithm, multiple wavelengths are necessary.

In respect of IgA, optimal results may be obtained by calculating the first derivative of absorbance at wavelengths of approximately 789 nm and 825 nm. In respect of β2-microglobulin, optimal results may be obtained by calculating the first derivative of absorbance at wavelengths of approximately 548 nm and 829 nm. In respect of CRP, optimal results may be obtained by calculating the first derivative of absorbance at wavelengths of approximately 661 nm and 679 nm.

The calibration algorithm developed for IgA based on 4 calibrators is as follows;

mgIL IgA=−3327114.33 (789 nm)+484270.80 (825 nm)−77.3 where (X nm) is the first derivative of the absorbance at the wavelength specified.

The calibration algorithm developed for β2-microglobulin based on 7 calibrators is as follows:

mg/L β2-microglobulin=−33648.79 (548 nm)+36556.81 (829 nm)+2.3

where (X nm) is the first derivative of the absorbance at the wavelength specified.

The calibration algorithm developed for CRP based on 9 calibrators is as follows:

mgL CRP=−1813682.71 (661 nm)+1808677.58 (679 nm)+9.8

where (X nm) is the first derivative of the absorbance at the wavelength specified.

It will be understood that several calibration algorithms can be developed for each protein, using an apparatus described for measuring specimen integrity.

The protein measurements are based on the principle of immunoturbidimetry, ie., generation of antibody-antigen complexes or immunocomplexes which cause turbidity. The “absorbance” generated is due to light scattering caused by the immunocomplexes, therefore endogenous turbidity or true absorbances caused by interferents in the sample will falsely elevate the signals. To demonstrate how interferents are dealt with, an aqueous solution of 2 g/L IgA was mixed with IL to provide 4 different samples with 1 g/L IgA and variable amounts of IL, i.e. from 0 to 4 g/L. Separate algorithms were developed for IgA with and without zero time correction.

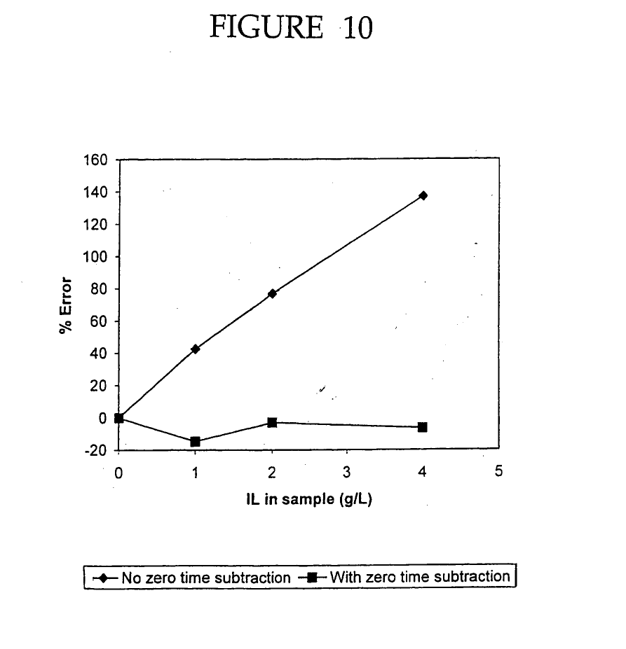

The error in the predicted results with and without zero time subtraction is shown in FIG. 10. This invention is different from the current art because multiple long wavelengths are used, and because of the small absorbances caused by the immunocomplexes at those wavelengths, endogenous interferents must be compensated for. This compensation cannot be performed using the raw absorbance due to the effect of small air bubbles and imprecise absorbance produce by disposable dispensing tips, but can be performed effectively by using the 1st derivative of the absorbance. As long as the first derivative of absorbance is employed, multiple wavelengths are necessary, even if the calibration algorithm uses a single first derivative of absorbance.

While the invention has been particularly shown and described with reference to preferred embodiments, it will be understood by those skilled in the art that various other changes in form, and detail may be made without departing from the scope of the invention.

I claim:

1. A method for determining the concentration of one or more plasma proteins in a sample by immunoturbidimetry in a blood analyzer, said method comprising the steps of:
filling a disposable dispensing tip with the sample;
sealing a first end of said disposable tip;
adding a reagent to an open second send of said disposable tip with a second tip capable of insertion into said open end;
placing said disposable tip into a heated cavity;
radiating the sample in said disposable tip with a source that emits a beam of radiation;
sensing the radiation emitted therefrom; and
correlating the concentration of said one or more proteins in said sample to the sensor response from the sample.
